(19) United States
(12) Patent Application Publication
(10) Pub. No.: US 2023/0268043 A1
(43) Pub. Date: Aug. 24, 2023

(54) THERAPEUTIC APPLICATION DISTRIBUTION SYSTEM, THERAPEUTIC APPLICATION DISTRIBUTION METHOD, THERAPEUTIC APPLICATION DISTRIBUTION PROGRAM, AND TERMINAL

(71) Applicant: CureApp, Inc., Tokyo (JP)

(72) Inventors: Shin Suzuki, Tokyo (JP); Ryuhei So, Tokyo (JP); Yutaka Kohada, Tokyo (JP)

(21) Appl. No.: 18/306,588

(22) Filed: Apr. 25, 2023

Related U.S. Application Data
(63) Continuation of application No. PCT/JP2021/034152, filed on Sep. 16, 2021.

(30) Foreign Application Priority Data
Oct. 26, 2020 (JP) 2020-179114

(51) Int. Cl. G16H 20/00; G16H 10/60; G16H 80/00

(52) U.S. Cl. CPC G16H 20/00; G16H 10/60; G16H 80/00

(57) ABSTRACT

A therapeutic application distribution system, which can be operated in a state suitable for a patient even in the initial execution of a therapeutic application, comprises: a server which accepts an activation code request for executing a therapeutic application from an information processing terminal of a patient as a subject to be treated by the therapeutic application, generates an activation code on the basis of initial information on a disease to be treated of the patient entered by a medical worker in advance, and transmits the activation code to the information processing terminal of the patient; and the information processing terminal which executes, by using the application code received from the server, the therapeutic application in a state where the initial information is applied.

### TECHNICAL FIELD

This disclosure relates to a therapeutic application distribution system for managing therapeutic applications, and a management method, management program thereof, and a terminal.

### BACKGROUND

Therapeutic applications have been developed to improve health-related behaviors of users, treat diseases, and manage their physical conditions recently (e.g. refer to Japanese Laid-Open Patent Publication No. 2001-092876). A therapeutic application corresponds to an application that records daily activities of a patient and notifies the patient of messages indicating what the patient is expected to perform to promote improvement of the conditions of the disease of the patient on the basis of the activities of the patient. According to JP 2001-092876A, on the basis of the data collected from individuals, messages are provided to the individuals to improve poor health behaviors.

In this regard, many of such therapeutic applications are only effective when used continuously by patients. On the other hand, as therapeutic applications, if the treatment continues to be performed, it can be effective based on the contents of the treatment and the changes of the treatment over time. However, such applications have a problem that they do not know the conditions of the patient at the time of initial execution and may fail to suggest appropriate treatment.

It could, therefore, be helpful to provide a therapeutic application distribution system, a therapeutic application distribution method, and a therapeutic application distribution program for distributing a therapeutic application that allows a patient to continue to use.

### SUMMARY

A therapeutic application distribution system includes: a server and an information processing terminal. The server includes: a first reception unit that receives, from an information processing terminal of a patient to be treated by a therapeutic application, user identification information identifying the patient, an application ID identifying the therapeutic application, and request information requesting an activation code to execute the therapeutic application; an acceptance unit that accepts input of initial information on the user identification information and a disease to be treated by the therapeutic application from a medical worker, and associates and stores the user identification information with the initial information in a storage; a generator that generates an activation code for executing the therapeutic application corresponding to the user identification information; and a first transmission unit that transmits the activation code generated by the generator to the information processing terminal of the patient. The information processing terminal includes: an application acquisition unit that acquires a therapeutic application in an initial state; a second transmission unit that transmits to the server the user identification information, the application ID identifying the therapeutic application acquired by the application acquisition unit, and the request information requesting the activation code to execute the therapeutic application; a second reception unit that receives the activation code from the server; an execution unit that executes the therapeutic application; and an output unit that outputs information based on the therapeutic application. The execution unit executes the therapeutic application with the initial information applied based on input of the activation code at an initial execution of the therapeutic application.

The initial information may include an age of the patient, the therapeutic application may make a suggestion for treatment of the disease, and the output unit may output a suggestion based on the age of the patient as information based on the therapeutic application.

The initial information may include gender of the patient, the therapeutic application may output a suggestion for treatment of the disease via one of a plurality of characters, and the output unit may output the suggestion as information based on the therapeutic application by a character suitable for the gender of the patient among the characters.

The initial information may include historical information on treatment for the disease of the patient, and the output unit may output information indicating a history of the treatment of the patient based on the history information as information based on the therapeutic application.

The output unit may output, as information based on the therapeutic application, a suggestion for treatment in accordance with a progress of the disease based on the historical information of the patient.

The historical information may be an electronic medical record of the patient.

The initial information may be information on personality of the patient, and the output unit may output a suggestion based on the personality of the patient as information based on the therapeutic application.

The server may include a provision unit that provides an input form for requesting the activation code to the information processing terminal, the input form may include a questionnaire for the patient, the second transmission unit may further transmit a response of the patient to the questionnaire, the first reception unit may further receive the response of the patient, and the initial information may include information based on the response of the patient to the questionnaire as information on the personality of the patient.

The initial information may include information on a suggestion for treatment performed on the patient by the medical worker, and the output unit may output a suggestion the same as the suggestion as information based on the therapeutic application.

A server includes: a reception unit that receives, from a terminal of a patient to be treated by a therapeutic application, user identification information identifying the patient, an application ID identifying the therapeutic application, and request information requesting an activation code to execute the therapeutic application; an acceptance unit that accepts input of initial information on the user identification information and a disease to be treated by the therapeutic application from a medical worker, and associates and stores the user identification information with the initial information in a storage; a generator that generates an activation code for executing the therapeutic application corresponding to the user identification information; and a transmission unit that transmits the activation code generated by the generator to a terminal of the patient.

An information processing terminal includes: an application acquisition unit that acquires a therapeutic application in an initial state; a second transmission unit that transmits to a server user identification information, an application ID identifying the therapeutic application acquired by the application acquisition unit, and request information requesting an activation code to execute the therapeutic application; a second reception unit that receives the activation code from the server; an execution unit that executes the therapeutic application; and an output unit that outputs information based on the therapeutic application. The activation code is associated with the user identification information, the user identification information is associated with initial information on information on a patient indicated by the user identification information and a disease to be treated by the therapeutic application, and the execution unit executes the therapeutic application with the initial information applied based on the input of the activation code at a first execution of the therapeutic application.

A therapeutic application distribution method includes: an application acquisition step of acquiring a therapeutic application in an initial state by an information processing terminal; a first transmission step of transmitting, by the information processing terminal, to a server user identification information, an application ID identifying a therapeutic application acquired in the application acquisition step, and request information requesting an activation code to execute the therapeutic application; a first reception step of receiving, by the server from the information processing terminal of a patient to be treated by the therapeutic application, user identification information identifying the patient, an application ID identifying the therapeutic application, and request information requesting an activation code to execute the therapeutic application; an acceptance step of accepting, by the server, input of initial information on the user identification information and a disease to be treated by the therapeutic application from a medical worker, and associating and storing the user identification information with the initial information in a storage; a generation step of executing, by the server, the therapeutic application corresponding to the user identification information; a second transmission step of transmitting, by the server, the activation code generated in the generation step to the information processing terminal of the patient; a second reception step of receiving, by the information processing terminal, the activation code from the server; an execution step of executing, by the information processing terminal, the therapeutic application; and an output step of outputting, by the information processing terminal, information based on the therapeutic application. In the execution step, the therapeutic application is executed with the initial information applied based on the input of the activation code at an initial execution of the therapeutic application.

A therapeutic application distribution method performed by a server includes: a reception step of receiving, from a terminal of a patient to be treated by a therapeutic application, user identification information identifying the patient, an application ID identifying the therapeutic application, and request information requesting an activation code to execute the therapeutic application; an acceptance step of accepting input of initial information on the user identification information and a disease to be treated by the therapeutic application from a medical worker, and associating and storing the user identification information with the initial information in a storage; a generation step of executing the therapeutic application corresponding to the user identification information; and a transmission step of transmitting the activation code generated in the generation step to the terminal of the patient. A therapeutic application execution method performed by a terminal includes: an application acquisition step of acquiring a therapeutic application in an initial state; a second transmission step of transmitting to a server user identification information, an application ID identifying the therapeutic application acquired in the application acquisition step, and request information requesting an activation code to execute the therapeutic application; a second reception step of receiving the activation code from the server; an execution step of executing the therapeutic application; and an output step of outputting information based on the therapeutic application. The activation code is associated with the user identification information, the user identification information is associated with initial information on information on a patient indicated by the user identification information and a disease to be treated by the therapeutic application, and in the execution step, the therapeutic application is executed with the initial information applied based on input of the activation code at an initial execution of the therapeutic application.

A therapeutic application distribution program causes a computer of a server to embody: a reception function that receives, from a terminal of a patient to be treated by a therapeutic application, user identification information identifying the patient, an application ID identifying the therapeutic application, and request information requesting an activation code to execute the therapeutic application; an acceptance function that accepts input of initial information on the user identification information and a disease to be treated by the therapeutic application from a medical worker, and associates and stores the user identification information with the initial information in a storage; a generation function that executes the therapeutic application corresponding to the user identification information; and a transmission function that transmits the activation code generated by the generation function to the terminal of the patient.

A therapeutic application execution program causes a computer of a terminal to embody: an application acquisition function that acquires a therapeutic application in an initial state; a second transmission function that transmits to a server user identification information, an application ID identifying the therapeutic application acquired by the application acquisition function, and request information requesting an activation code to execute the therapeutic application; a second reception function that receives the activation code from the server; an execution unit that executes the therapeutic application; and an output function that outputs information based on the therapeutic application. The activation code is associated with the user identification information, the user identification information is associated with initial information on information on a patient indicated by the user identification information and a disease to be treated by the therapeutic application, and the execution function executes the therapeutic application with the initial information applied based on input of the activation code at an initial execution of the therapeutic application.

The therapeutic application distribution system reflects patient-specific information on a therapeutic application when a patient uses the therapeutic application for the first time so that even when the therapeutic application is used for the first time, it can be operated in a condition suitable for the conditions of the disease of the patient.

### BRIEF DESCRIPTION OF THE DRAWINGS

FIG. 1 is a system diagram illustrating an overview of a therapeutic application management system.

FIG. 2 is a block diagram illustrating an example configuration of a management server.

FIG. 3 is a block diagram illustrating an example configuration of a user terminal.

FIG. 4 is a block diagram illustrating an example configuration of a distribution server.

FIG. 5 is a block diagram illustrating an example configuration of a medical worker terminal.

FIG. 6 is a block diagram illustrating an example configuration of an operation server.

FIG. 7 is a schematic data diagram illustrating an example of data structure of management information.

FIG. 8 is a sequence diagram illustrating examples of exchanges among devices related to the therapeutic application management system.

FIG. 9 is a flowchart illustrating an example of an operation of the management server.

FIG. 10 is a flowchart illustrating an example of an operation of a user terminal.

FIG. 11 is a flowchart illustrating an example of an operation of a distribution server.

FIG. 12 is a flowchart illustrating an example of an operation of a medical worker terminal.

FIG. 13 is a flowchart illustrating an example of an operation of the operation server.

FIG. 14A is a diagram illustrating an example of an initial display screen at the terminal when no initial information is provided.

FIG. 14B is a diagram illustrating an example of an initial display screen at the terminal when initial information is provided.

FIG. 15 shows an example of a chatbot selection table in accordance with a user.

FIG. 16 is a flowchart illustrating an example of an operation of a terminal that determines a chatbot that will respond to a user.

FIG. 17 shows an example of a table illustrating examples of responses in accordance with a status of the user.

FIG. 18 is a flowchart illustrating an example of an operation of a terminal showing an example of determining a suggestion method to the user.

FIGS. 19A to 19C show example screens of one of suggestions.

FIG. 20 is a sequence diagram illustrating examples of exchanges among devices related to a variation of the therapeutic application management system.

FIG. 21 is a flowchart illustrating an example of an operation of a variation of the medical worker terminal.

FIG. 22 is a flowchart illustrating an example of an operation of a variation of a user terminal.

FIG. 23 is a flowchart illustrating an example of an operation of a variation of a distribution server.

### DESCRIPTION OF THE REFERENCE NUMERALS

100: Management Server
110, 210, 310, 510: Reception Unit
120, 220, 320, 520: Input Unit
130, 230, 330, 530: Controller
131: Acquisition Unit
132: Registration Unit
133: Timekeeping Unit
134: Restriction Unit
140, 240, 340, 540: Storage
141: Management Information
150, 250, 350, 550: Output Unit
200: User Terminal
231: Application Execution Unit
300: Distribution Server
331: Distribution Unit
341: First Therapeutic Application Installer
342: Second Therapeutic Application Installer
400: Network
500: Medical Worker Terminal
531: Initial Information Transmission Unit

### DETAILED DESCRIPTION

Features, advantages, and technical and industrial significance of examples will be described below with reference to the accompanying drawings, in which like numerals denote like elements, and wherein:

With reference to the drawings, an information processing apparatus according to an example will be described in detail.

### Example

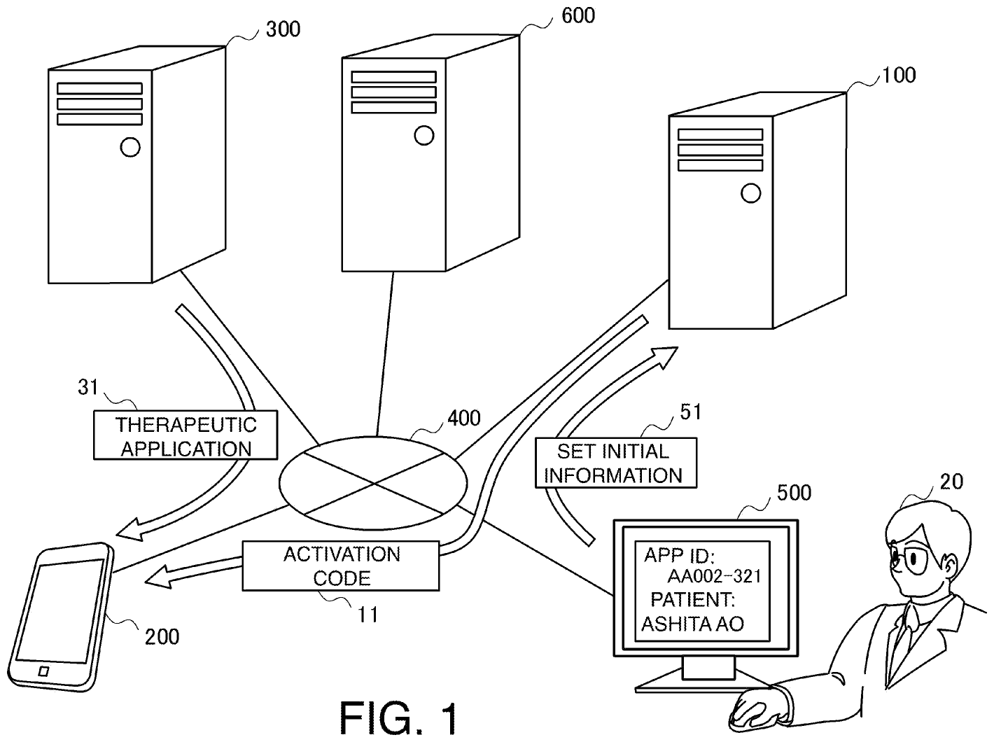

FIG. 1 is a system diagram illustrating an overview of the therapeutic application distribution system 1 according to a first example and shows an example of a system configuration thereof. As shown in FIG. 1, the therapeutic application distribution system 1 includes a management server 100 and a distribution server 300, and may include a user terminal 200, a medical worker terminal 500, and an operation server 600. The management server 100, user terminal 200, distribution server 300, medical worker terminal 500, and operation server 600 are connected to communicate with each other via network 400. Although one unit is shown for each device in FIG. 1, each terminal and each type of server is not limited to one unit, that is, multiple servers may be provided, each of which may have the same functions, or multiple servers may be configured to have the same functions as a single server.

The management server 100 corresponds to a server device, which manages therapeutic applications used by a patient that holds the user terminal 200, and issues an activation code to allow the patient to use the corresponding therapeutic application. Each activation code may be directed to any information that activates the corresponding therapeutic application, i.e., enables the therapeutic application to be used, and is not limited to the name of the activation code. As an example, the activation code may be called a passcode, passphrase and serial code, for example, and may be substituted. The distribution server 300 corresponds to a server device, which serves to distribute the therapeutic applications in initial states to the user terminal 200. The user terminal 200 corresponds to an information processing terminal held by the patient and may be embodied by a smartphone, tablet terminal, cell phone, and PC, for example. The medical worker terminal 500 corresponds to an information processing device used by medical workers. In the same manner as in the user terminal 200, the medical worker terminal may be embodied by a smartphone, tablet terminal, cell phone, and PC, for example. The operation server 600 corresponds to a server device, which provides the user terminal 200 of the patient with therapeutic information on the treatment of the patient when the therapeutic application is executed.

The therapeutic application may be directed to application software, which manages the daily conditions of the patient and provides guidance associated with personalized medical knowledge to each patient.

In addition, the medical worker can be any person involved in healthcare, for example, a physician, nurse, mentor, or pharmacist.

In the therapeutic application distribution system 1, initial information for operating the therapeutic application is entered or set up to be associated with existing data as initial information to the medical worker terminal 500 by the medical worker 20. The entered user (patient) information is transmitted to the management server 100 as initial information 51, and the management server 100 associates, registers and manages the information with each patient and each therapeutic application.

The patient, on the other hand, uses the user terminal 200 to download and use the therapeutic application distributed from the distribution server 300. In addition, in using the therapeutic application 31, the user terminal 200 receives the activation code 11 from the management server 100. The activation code 11 is associated with the initial information set by the medical worker 20 so that upon initial execution, the therapeutic application can be started with the information on the patient of the user terminal 200 entered. The management server 100 collects, stores, and manages various types of data accumulated as the patients use the therapeutic applications. When the user terminal 200 executes the therapeutic application, it can display therapeutic information provided by the management server 100 or the operation server 600 as information on the treatment of the patient of the user terminal 200.

Hereinafter, a description will be given below. The network 400 is directed to a network for interconnecting the various devices, including, for example, a wireless network or a wired network. Specifically, the network may include a wireless LAN (WLAN) or a wide area network (WAN), integrated service digital networks (ISDNs), wireless LANs, long term evolution (LTE), LTE-Advanced, 4th Generation (4G), 5th generation (5G), code division multiple access (CDMA), WCDMA (registered trademark), and Ethernet (registered trademark), for example. The network is not limited to these examples, but may include, for example, the public switched telephone network (PSTN), Bluetooth (registered trademark), and Bluetooth Low Energy (registered trademark), optical communication lines, asymmetric digital subscriber line (ADSL), satellite communication networks, or any other network. The network is sometimes referred to as a home network when it is provided in the residence of the user. The network may also include, for example, a narrow band IoT (NB-IoT) and enhanced machine type communication (eMTC). In addition, the NB-IoT and eMTC are wireless communication methods for IoT, which are low-cost, low-power, and long distance communication network. The network may also be a combination of these. The network may also include a plurality of different networks combining these examples. For example, the network may include a wireless network based on LTE and a wired network such as an intranet as a closed network.

### Example Configuration of Management Server 100

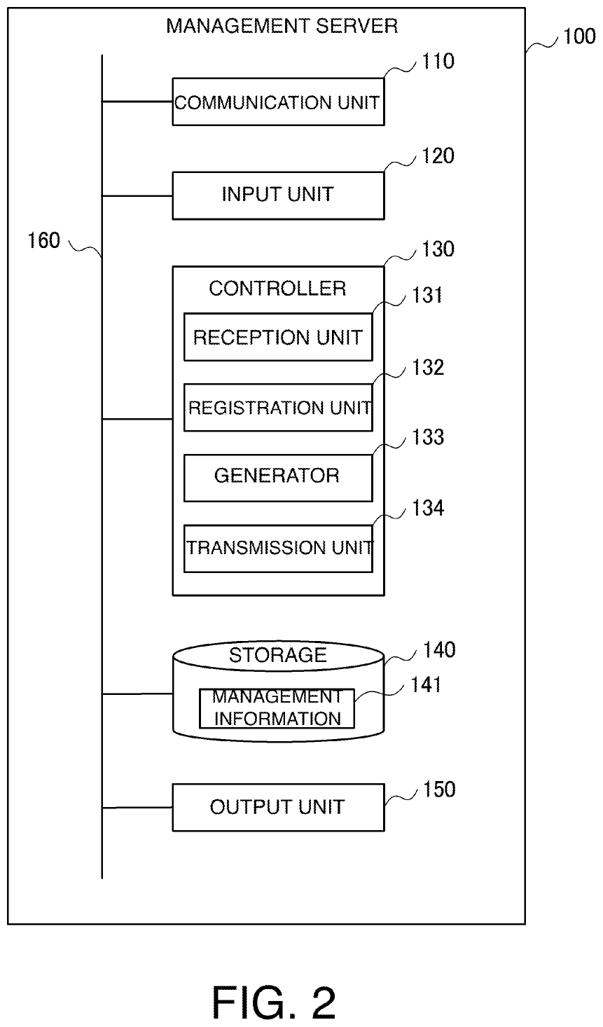

FIG. 2 is a block diagram illustrating an example configuration of the management server 100.

As shown in FIG. 2, the management server 100 includes a communication unit 110, an input unit 120, a controller 130, a storage 140, and an output unit 150. The communication unit 110, input unit 120, controller 130, storage 140, and output unit 150 may be configured to communicate with each other via bus 160.

The communication unit 110 is directed to a communication interface that executes communication with other devices. The communication unit 110 may communicate with other devices by any communication protocol, wired or wireless, if it is capable of communicating with other devices. The communication unit 110 communicates with the user terminal 200, the distribution server 300, the medical worker terminal 500, or the operation server 600 in accordance with the instructions from the controller 130. The communication unit 110 receives, for example, information indicating a therapeutic application, information on the patient that uses the therapeutic application, and initial information associated with the use of the therapeutic application from the medical worker terminal 500 and communicates them to the controller 130. Further, the communication unit 110, for example, receives request information requesting an activation code from the user terminal 200 and communicates it to the controller 130. Moreover, the communication unit 110 transmits, for example, activation codes associated with initial information to the user terminal 200 and restriction information to restrict the use of the therapeutic application in accordance with the instructions from the controller 130.

The input unit 120 is an input interface that accepts and communicates input from the operator of the management server 100, for example, to the controller 130. The input unit 120 may be embodied by soft keys such as a touch panel, or by hard keys. Alternatively, the input unit 120 may be a microphone for accepting voice input. The input unit 120 accepts input entered by the medical workers or operators of the management server 100, and communicates the input contents to the controller 130.

The storage 140 serves to store various programs and data needed by the management server 100 to operate. The storage 140 may be embodied by hard disk drive (HDD), solid state drive (SSD), and flash memory, for example. The storage 140 stores management information 141. The management information 141 corresponds to information for managing the therapeutic applications for the users. The details of the management information 141 will be described below.

The output unit 150 serves to output the specified information in accordance with the instructions from the controller 130. The output by the output unit 150 may be in the form of image or audio signals. In output based on the image signals, it may be supplied to a monitor connected to (or provided by) the management server 100. In the case of output based on the audio signals, it may be supplied to a speaker connected to (or provided by) the management server 100.

The controller 130 is directed to a processor that serves to control portions of the management server 100. The controller 130 may be embodied by a single core or by multiple cores.

The controller 130 includes a reception unit 131, a registration unit 132, a generator 133, and a transmission unit 134 as functions embodied by the controller 130.

The reception unit 131 receives information indicating the therapeutic application, user identification information identifying the user (patient) to whom the therapeutic application is prescribed, and initial information that can be applied when the therapeutic application is first used by that patient. The information indicating the therapeutic application may be identification information that identifies the therapeutic application, or it may be the application itself. These information may also be received separately if the controller 130 can recognize that they are associated with each other.

Further, the reception unit 131 receives request information requesting the activation code from the user terminal 200.

The registration unit 132 registers and associates the various information obtained by the reception unit 131 with each other in the management information 141. The updated management information 141 is stored in the storage 140 by the registration unit 132.

The generator 133 generates an activation code corresponding to the user identification information included in the request information and the application ID in accordance with the request information received by the reception unit 131. The generator 133 identifies whether the initial information corresponding to the user identification information included in the request information and application ID is registered in the management information 141. If the initial information is associated with them, the activation code associated with the initial information is generated. At this time, the generator 133 generates a patient ID for the management server 100 to manage the user (patient) corresponding to the user identification information and associates it with the user identification information and initial information to perform subsequent management. The activation code associated with the initial information is directed to a code that, when applied, enables the therapeutic application to be used with information on the corresponding patient entered during the initial execution of the therapeutic application. If the initial information corresponding to the user identification information included in the request information and the application ID has not been registered in the management information 141, the generator 133 generates an activation code with which no initial information is associated.

The transmission unit 134 transmits the activation code generated by the generator 133 to the user terminal 200 that has transmitted the request information requesting the activation code.

The example configuration of the management server 100 is as described above.

### Example Configuration of User Terminal 200

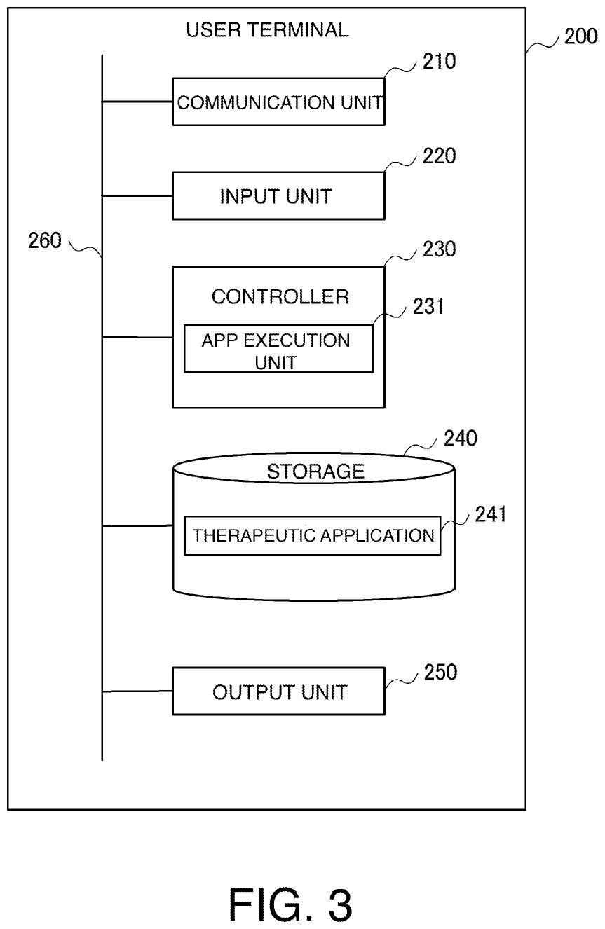

FIG. 3 is a block diagram illustrating an example configuration of a user terminal 200. As shown in FIG. 3, the user terminal 200 includes a communication unit 210, an input unit 220, a controller 230, a storage 240, and an output unit 250. The communication unit 210, input unit 220, controller 230, storage 240, and output unit 250 may be configured to communicate with each other via bus 260.

The communication unit 210 is directed to a communication interface that executes communication with other devices. The communication unit 210 may communicate with other devices by any communication protocol, wired or wireless, if it is capable of communicating with other devices. The communication unit 210 communicates with the management server 100 and distribution server 300 in accordance with the instructions from the controller 230.

The input unit 220 is an input interface that accepts and communicates input from the patient of the user terminal 200 to the controller 230. The input unit 220 may be embodied by soft keys such as a touch panel, or by hard keys. Alternatively, the input unit 220 may be a microphone for accepting voice input. The input unit 220 communicates the contents entered by the patient to the controller 230.

The controller 230 is directed to a processor that controls portions of the user terminal 20. The controller 230 may be embodied by a single core or by multiple cores. The controller 230 requests the request information requesting the therapeutic application to the distribution server 300 via the communication unit 210 in accordance with the input entered from the patient.

The controller 230 also includes an application execution unit 231 as a function embodied by the controller 230.

The application execution unit 231 executes the therapeutic application downloaded from the distribution server 300. The application execution unit 231 executes the therapeutic application, registers various information into the therapeutic application in accordance with the input from the patient, and obtains and displays information on the treatment from the management server 100 or the operation server 600 that stores the therapeutic information. The application execution unit 231 requests an activation code from the management server 100 when executing the therapeutic application. The application execution unit 231 then accepts the input of the activation code and executes the therapeutic application. If the activation code corresponds to an activation code associated with the initial information, even at the time of initial execution, the application execution unit 231 provides the patient of the user terminal 200 with the therapeutic application including the information associated with the patient. If the activation code does not correspond to the activation code associated with the initial information, the application execution unit 231 provides the patient with the therapeutic application in its initial state.

The storage 240 stores various programs and data required by the user terminal 200 to operate. The storage 140 may be embodied by hard disk drive (HDD), solid state drive (SSD), and flash memory, for example. FIG. 3 shows an example where the storage 240 stores the therapeutic applications 241. If a patient suffers from several different diseases, different therapeutic applications may be stored in the storage 240 for corresponding diseases.

The output unit 250 outputs the specified information in accordance with the instructions from the controller 230. The output by the output unit 250 may be in the form of image or audio signals. In the output based on the image signals, it may be supplied to a monitor that the user terminal 200 includes (or is connected to the user terminal 200). Further, in the case of the output based on the audio signals, it may be supplied to a speaker that the user terminal 200 includes (or is connected to the user terminal 200). The output unit 250, for example, displays information provided by the therapeutic application on the monitor of the user terminal 200 as the application execution unit 231 executes the therapeutic application.

The example configuration of the user terminal 200 is as described above.

### Example Configuration of Distribution Server 300

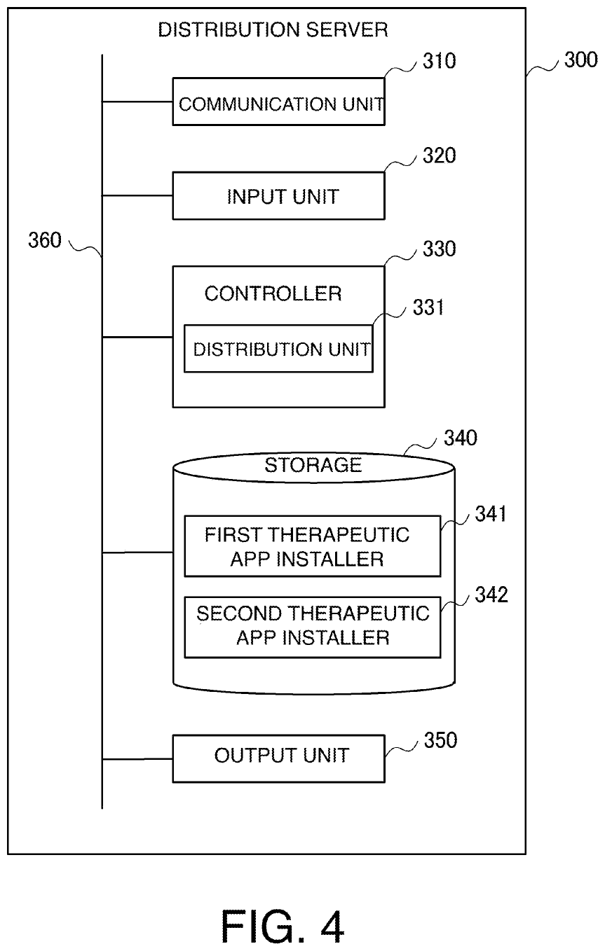

FIG. 4 is a block diagram illustrating an example configuration of a distribution server 300. The distribution server 300 is directed to a download server that stores the therapeutic applications designated by the management server 100 and distributes them upon a request from the user terminal 200.

As shown in FIG. 4, the distribution server 300 includes a communication unit 310, an input unit 320, a controller 330, a storage 340, and an output unit 350. The communication unit 310, input unit 320, controller 330, storage 340, and output unit 350 may be configured to communicate with each other via bus 360.

The communication unit 310 is directed to a communication interface that serves to execute communication with other devices. The communication unit 310 may communicate with other devices by any communication protocol, wired or wireless, if it is capable of communicating with other devices. The communication unit 310 communicates with the management server 100 and user terminal 200 in accordance with the instructions from the controller 130.

The input unit 320 is an input interface that accepts and communicates input from the operator of the distribution server 300, for example, to the controller 330. The input unit 320 may be embodied by soft keys such as a touch panel, or by hard keys. Alternatively, the input unit 320 may be a microphone for accepting voice input.

The controller 330 is directed to a processor that controls portions of the user terminal 20. The controller 330 may be embodied by a single core or by multiple cores.

The controller 330 includes a distribution unit 331 as a function embodied by the controller 330.

The distribution unit 331 identifies the installer of the therapeutic application indicated by the request information communicated via the communication unit 310 and reads it from the storage 340. The read-out therapeutic application installer is then transmitted via the communication unit 310 to the user terminal 200 that has transmitted the request information.

The storage 340 stores various programs and data needed by the distribution server 300 to operate. The storage 140 may be embodied by hard disk drive (HDD), solid state drive (SSD), and flash memory, for example. The storage 340 stores all the therapeutic applications (installers of the applications) requested by the management server 100. FIG. 4 shows an example of storing a first therapeutic application installer 341 and a second therapeutic application installer 342. The first therapeutic application installer 341 and the second therapeutic application installer 342 correspond to applications for treating different diseases from each other, and may be directed to, for example, a smoking cessation application, an alcohol cessation application, or a diabetes application. Although the example shown here is an example of two installers being stored, the number of installers is not limited to two, and installers for various disease-specific applications may be stored.

The output unit 350 serves to output the specified information in accordance with the instructions from the controller 330. The output by the output unit 350 may be in the form of image or audio signals. In the output based on the image signals, it may be supplied to a monitor connected to (or provided by) the distribution server 300. Further, in the output based on the audio signals, it may be supplied to a speaker that the distribution server 300 includes (or is connected to the distribution server 300).

The example configuration of the distribution server 300 is as described above.

### Example Configuration of Medical Worker Terminal 500

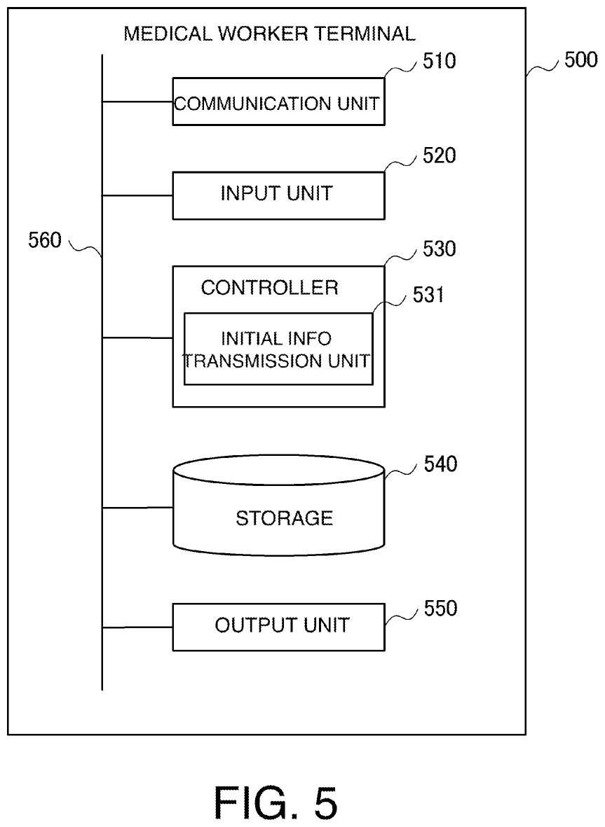

FIG. 5 is a block diagram illustrating an example configuration of the medical worker terminal 500. The medical worker terminal 500 corresponds to an information processing terminal used by medical workers. As shown in FIG. 5, the medical worker terminal 500 includes a communication unit 510, an input unit 520, a controller 530, a storage 540, and an output unit 550. The communication unit 510, input unit 520, controller 530, storage 540, and output unit 550 may be configured to communicate with each other via bus 560.

The communication unit 510 is directed to a communication interface that executes communication with other devices. The communication unit 510 may communicate with other devices by any communication protocol, wired or wireless, if it is capable of communicating with other devices. The communication unit 510 communicates with the management server 100, the distribution server 300, and the operation server 600 in accordance with the instructions from the controller 530.

The input unit 520 is an input interface that accepts and communicates input from the patient of the user terminal 200 to the controller 530. The input unit 520 may be embodied by soft keys such as a touch panel, or by hard keys. Alternatively, the input unit 520 may be a microphone for accepting voice input. The input unit 520 communicates the contents entered by the patient to the controller 530.

The input unit 520 accepts, from the medical worker, the input of the initial information on the patient that will be using the therapeutic application and communicates it to the controller 530. The initial information may be any information on the corresponding patient such as the name, age, and gender of the patient, and one or more of the followings: personality, course of treatment to date, stage of disease, and information that identifies the storage location of the server device that stores such information. Specifically, the input unit 520 accepts and communicates the input to the controller 530, including the application ID that specifies the therapeutic application with which the initial information is associated, user identification information indicating the patient that uses the application, and the initial information of the therapeutic application.

The controller 530 is directed to a processor that controls portions of the medical worker terminal 500. The controller 530 may be embodied by a single core or by multiple cores.

The controller 530 includes an initial information transmission unit 531 as a function embodied by the controller 530.

The initial information transmission unit 531 transmits the application ID, the user identification information indicating the user (patient) using the application, and the initial information entered by the medical worker via the input unit 520 to the management server 100 via the communication unit 510.

The storage 540 stores various programs and data needed by the medical worker terminal 500 to operate. The storage 540 may be embodied by hard disk drive (HDD), solid state drive (SSD), and flash memory, for example.

The output unit 550 outputs the specified information in accordance with the instructions from the controller 530. The output by the output unit 550 may be in the form of image or audio signals. In the output based on the image signals, it may be supplied to a monitor connected to (or provided by) the medical worker terminal 500. Further, in the output based on the audio signals, it may be supplied to a speaker that the medical worker terminal 500 includes (or is connected to the medical worker terminal 500). The output unit 550 may, for example, display the initial information on the patient in the therapeutic application entered by the medical worker as image data.

The example configuration of the medical worker terminal 500 is as described above.

### Example Configuration of Operation Server 600

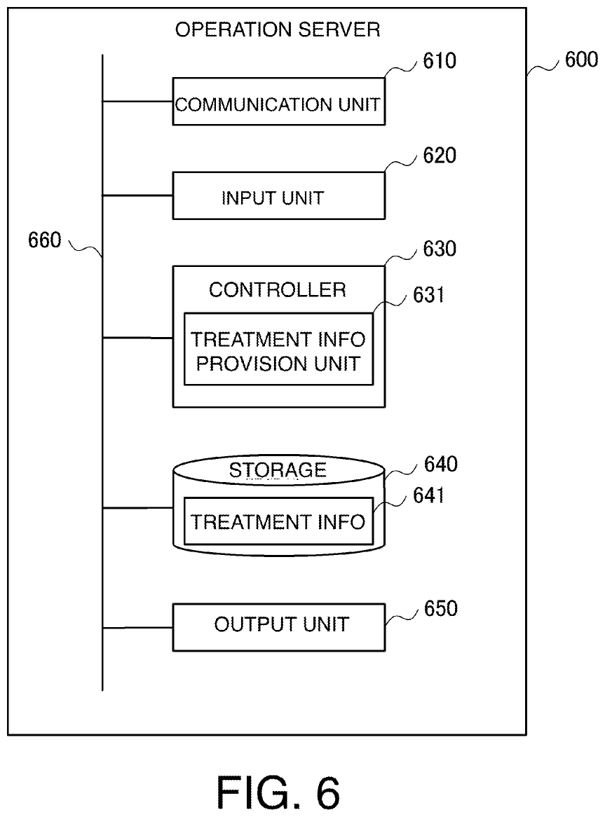

FIG. 6 is a block diagram illustrating an example configuration of an operation server 600. The operation server 600 corresponds to an information processing device (server) that manages and operates information on the treatment of the patients that use the therapeutic applications. The operation server 600 may, as information on the treatment of the patient to be managed by the operation server 600, be set up for the diseases of each patient, or for each treatment facility (hospital, and treatment center, for example) where the patient is treated, or for each operation company that provides therapeutic applications, or it may be managed together on a single server. As shown in FIG. 6, the operation server 600 includes a communication unit 610, an input unit 620, a controller 630, a storage 640, and an output unit 650. The communication unit 610, input unit 620, controller 630, storage 640, and output unit 650 may be configured to communicate with each other via bus 660.

The communication unit 610 is directed to a communication interface that serves to execute communication with other devices. The communication unit 610 may communicate with other devices by any communication protocol, wired or wireless, if it is capable of communicating with other devices. The communication unit 610 communicates with the management server 100 and user terminal 200 in accordance with the instructions from the controller 630.

The input unit 620 is an input interface that accepts and communicates input from the patient of the user terminal 200 to the controller 630. The input unit 620 may be embodied by soft keys such as a touch panel, or by hard keys. Alternatively, the input unit 620 may be a microphone for accepting voice input. The input unit 620 communicates the contents entered by the patient to the controller 630.

The input unit 620 accepts and communicates the input of the information on the patient to be treated from a medical worker (mainly physicians) to the controller 630. The information on the patient to be treated may be any information on the patient, including the medical conditions of the patient, drugs prescribed to the patient, and course of the treatment by the medical worker. Specifically, the input unit 620 accepts, and communicates input of a patient ID indicating the patient that is the subject of the information to be entered and information on the patient indicated by the patient ID to the controller 630.

The controller 630 is directed to a processor that controls portions of the operation server 600. The controller 630 may be embodied by a single core or by multiple cores. When the controller 630 receives a patient ID and information on the patient via the input unit 620, it associates and records the information on the patient with the communicated patient ID as treatment information 641 in the storage 640.

The controller 630 includes a treatment information provision unit 631 as a function embodied by the controller 630.

When the treatment information provision unit 631 receives a request for transmission of the treatment information from the user terminal 200 via the communication unit 610, it identifies from the treatment information 641 the information on the patient associated with the patient ID indicated by the request for transmission of the treatment information. The treatment information provision unit 631 transmits the identified treatment information (information on the patient) to the user terminal 200 via the communication unit 610.

The storage 640 stores various programs and data needed by the operation server 600 to operate. The storage 640 may be embodied by hard disk drive (HDD), solid state drive (SSD), and flash memory, for example. The storage 640 stores treatment information 641 on the treatment of the patient. The treatment information is associated with the patient ID of each patient and information on the patient. The treatment information may be an electronic medical record. The treatment information may also include information on the behavior associated with the treatment of the patient himself/herself entered by the patient. Further, the treatment information may be associated with the application ID of the therapeutic application used by the patient.

The output unit 650 outputs the specified information in accordance with the instructions from the controller 630. The output by the output unit 650 may be in the form of image or audio signals. In the case of the output based on the image signals, it may be supplied to a monitor connected to (or provided by) the operation server 600. Further, in the case of the output based on the audio signals, it may be supplied to a speaker that the operation server 600 includes (or is connected to the operation server 600). The output unit 650 may, for example, output the treatment information of the patient.

The example configuration of the operation server 600 is as described above.

### Data

Hereinafter, the management information 141 will be described. As described above, the information is used by the management server 100 to manage each therapeutic application used by each patient. Further, the management information 141 may be shared with the operation server 600.

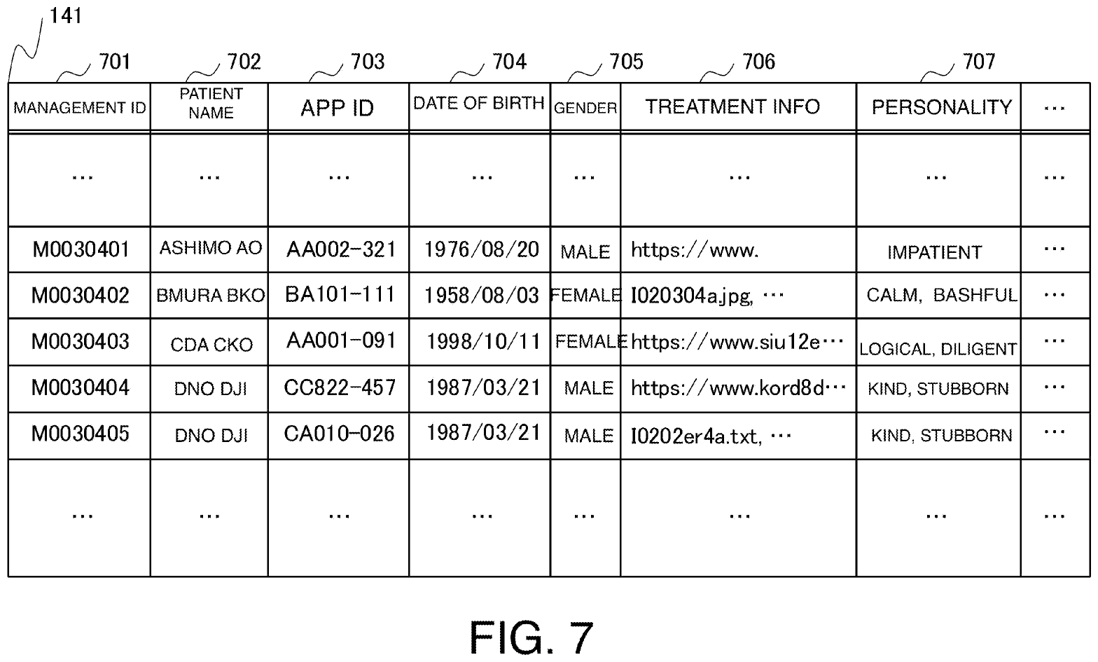

FIG. 7 is a schematic data diagram illustrating an example of data structure of the management information 141. As shown in FIG. 7, the management information 141 is directed to information in which management ID 701, patient name 702, application ID 703, date of birth 704, gender 705, treatment information 706, and personality 707 are associated with each other.

The management ID 701 corresponds to identification information for management that is assigned by the management server 100 for convenience to manage each therapeutic application used by each patient contained in the management information 141.

The patient name 702 corresponds to information indicating the name of the patient using the therapeutic application. The patient name 702 may be an identifier capable of uniquely identifying the patient rather than the patient name.

The application ID 703 corresponds to identification information capable of uniquely identifying the therapeutic application used by the patient indicated by the corresponding patient name 702.

The date of birth 704 corresponds to information indicating the date of birth of the patient indicated by the corresponding patient name 702. The date of birth 704 can be used as information capable of identifying the age of the patient.

The gender 705 corresponds to information indicating the gender of the patient indicated by the corresponding patient name 702.

The treatment information 706 corresponds to information indicating the actual data of information on the treatment that can be confirmed as the therapeutic application indicated by the application ID 703, which is used by the patient indicated by the corresponding patient name 702, or the address of the device capable of confirming the actual data. The device capable of confirming the actual data here corresponds to a server device that stores information such as medical examination information by the medical institution where the patient is actually receiving treatment, for example, where the server device of the medical institution, for example, is associated with the management server 100. When all of the treatment information 706 is operated and managed by the operation server 600, it need not be registered in the management information 141. Further, the treatment information 706 and the treatment information 641 stored by the operation server 600 may be the same or different to each other. One example where the treatment information is different from each other may be, for example, such that the management server 100 stores information on the treatment of the patient for smoking cessation, and the operation server 600 stores information on the treatment for diabetes of the patient in contrast.

The personality 707 corresponds to information indicating the personality of the patient indicated by the corresponding patient name 702. The personality 707 basically corresponds to information entered by a medical worker that has been involved in the treatment of the patient indicated by the corresponding patient name 702, but it may also correspond to information entered by the patient himself/herself. Further, the personality 707 may correspond to information inferred from the course of using the therapeutic application. Moreover, the personality 707 may be masked data that the patient himself/herself cannot identify.

If the same patient is using different therapeutic applications, they are registered separately in the management information 141. In addition to these information, the management information 141 may also contain information such as the address of the user terminal 200 of each patient and telephone number as contact information.

### Operation

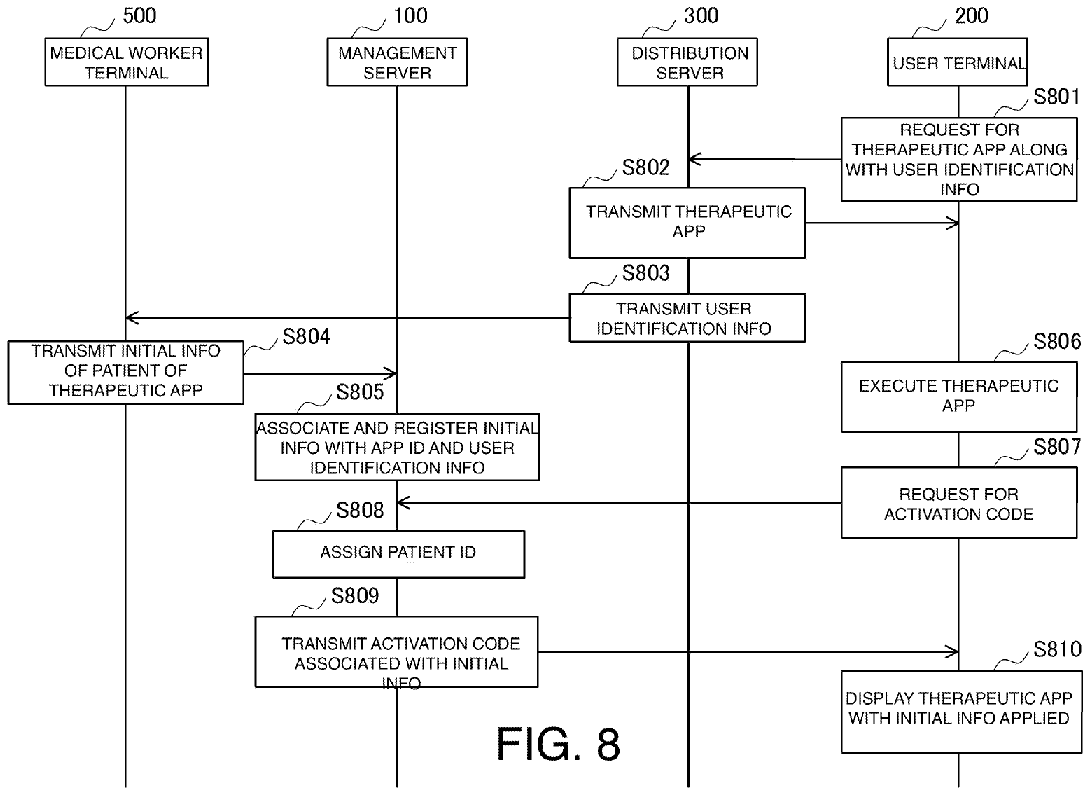

FIG. 8 is a sequence diagram illustrating examples of exchanges among devices related to the therapeutic application distribution system 1.

As shown in FIG. 8, the user terminal 200 transmits request information requesting a therapeutic application along with the user information to the distribution server 300 in accordance with the input from the patient at the user terminal 200 (step S801). The user information corresponds to information capable of identifying the user of the user terminal 200, and it is sufficient if the medical worker can recognize which user it is directed to.

The distribution server 300 receives the request information from the user terminal 200 and transmits the therapeutic application specified in the request information to the user terminal 200 (step S802).

The distribution server 300 also transmits user information indicating the user corresponding to the user terminal 200 that has requested the therapeutic application and an application ID indicating the requested therapeutic application to the medical worker terminal 500 (step S803). The process of step S803 may be performed from the user terminal 200 to the medical worker terminal 500, that is, the user information and application ID may be transmitted from the user terminal 200 to the medical worker terminal 500.

The medical worker terminal 500 transmits the initial information of the therapeutic application entered by the medical worker to the management server 100 (step S804). The initial information is directed to information related to the user (patient) that uses the therapeutic application. The initial information may include, for example, the name, age, gender, weight, blood type, and anamnesis of the user (patient), and may also be directed to information on the conditions of the user with respect to the disease that the therapeutic application is treating. Alternatively, it may be directed to the electronic medical record of the user (patient) in the treatment of a disease related to the therapeutic application, or it may contain information on the address of the operation server 600 where the electronic medical record is stored.

The management server 100 associates and registers the initial information with the therapeutic application indicated by the application ID and user identification information received from the medical worker terminal 500 (step S805).

When the user terminal 200 receives the therapeutic application from the distribution server 300, it executes the therapeutic application in accordance with the instructions from the patient (step S806). When the user terminal 200 executes the therapeutic application, it requests an activation code to the management server 100 to execute the therapeutic application (step S807). At this time, the user terminal 200 also transmits the user identification information that it has transmitted when it has requested the therapeutic application to the distribution server 300.

When the management server 100 accepts the request for the activation code, it assigns a patient ID to the received user identification information to uniquely identify the patient from other patients for management by the management server 100 (step S808). Upon accepting the request for the activation code from the user terminal 200, the management server 100 transmits the activation code associated with the initial information on the patient of the user terminal 200 (step S809).

The patient of the user terminal 200 checks and enters the activation code transmitted from the management server 100. Upon accepting the activation code, the therapeutic application displays content related to the treatment with the initial information applied (step S809). The treatment-related content to be displayed here may be obtained from the treatment information 706 stored by the management server 100 or from the predetermined operation server 600. Alternatively, or as initial information, the content may correspond to information that is associated on the management server 100.

The process of step S807 in FIG. 8 may be executed before the process of step S806 such that the content may be obtained before the therapeutic application is executed. In this example, the assignment of the patient ID may be performed at a time point in which the activation code is authenticated. In FIG. 8, the patient ID is assigned at the time point when the activation code is requested, but it may be performed at the time point when the user terminal 200 accepts the input of the activation code and the management server 100 successfully authenticates it as described above.

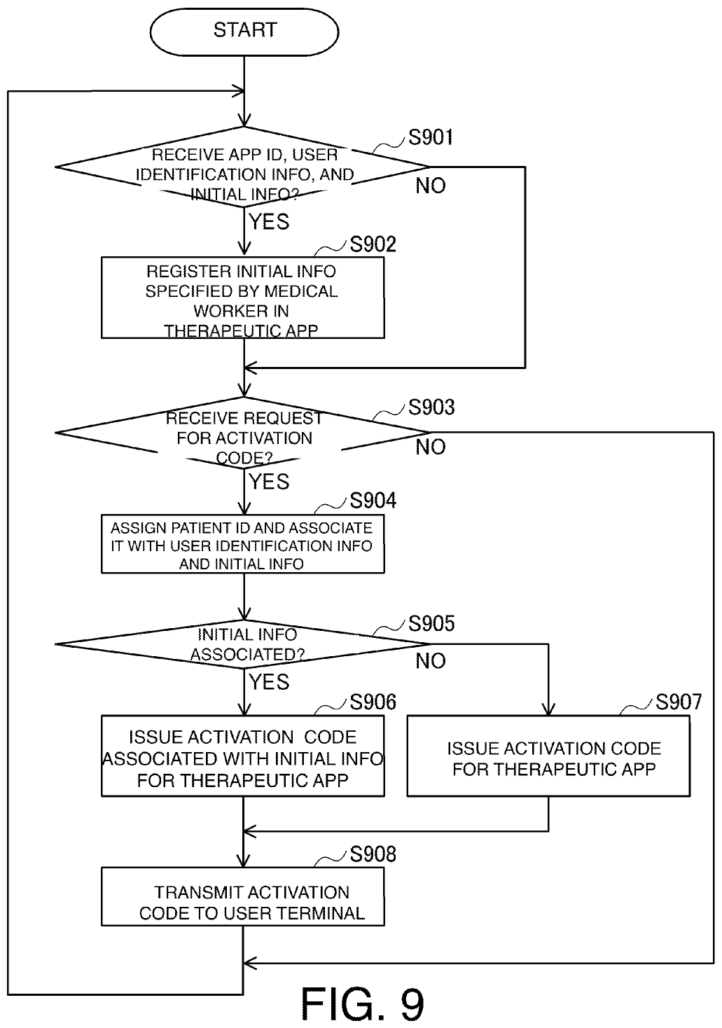

FIG. 9 is a flowchart illustrating an example of the operations of the management server 100 to embody the exchanges shown in FIG. 8.

When the communication unit 110 of the management server 100 receives a signal, it checks whether the signal corresponds to an application ID, user identification information, and initial information. If the communication unit 110 receives each piece of information including the initial information (YES in step S901), it communicates each received information including the initial information to the controller 130. If the received signal does not correspond to the initial information (NO in step S901), the process moves to step S903.

When the controller 130 receives each piece of information including the initial information, the registration unit 132 associates and registers the initial information with the application ID and the patient ID in the management information 141 (step S902). If the initial information is directed to various personal information related to the patient (e.g., name, age, gender, and personality), the registration unit 132 registers the information in the corresponding fields, and if the information indicates cloud storage where the information on the treatment related to the patient is stored, the registration unit 132 registers the information in the treatment information 706 of the management information 141. This causes the initial information to be associated and managed with the corresponding therapeutic application in the management server 100.

When the communication unit 110 receives a signal, it checks whether the signal is directed to a request for an activation code for a therapeutic application. If the communication unit 110 receives a signal requesting an activation code (YES in step S903), it communicates the received signal to the controller 130. If the request for an activation code has not been received (NO in step S903), the process moves to step S901.

When the controller 130 accepts a request for an activation code, it identifies the application ID indicating the therapeutic application included in the request and the user identification information. A patient ID is then assigned to the user indicated by the received user identification information to uniquely identify and manage the user on the management server 100. The controller 130 associates and stores the generated patient ID with the user identification information in the storage 140 (step S904).

The controller 130 checks whether the initial information is associated with the received user identification information by referring to the storage 140 (step S905). If the initial information is associated (YES in step S905), the generator 133 of the controller 130 generates an activation code for the identified therapeutic application, with which code the initial information is associated (step S906). If the initial information is not associated with the user identification information (NO in step S905), an activation code without associated initial information is generated, that is, an activation code for activating the therapeutic application in the initial state is generated (step S907).

The transmission unit 134 transmits the activation code generated by the generator 133 to the user terminal 200 with the identified patient ID via the communication unit 110 (step S908).

The processes in steps S901 and S902 and the processes in steps S903 through S908 may be executed in parallel with each other.

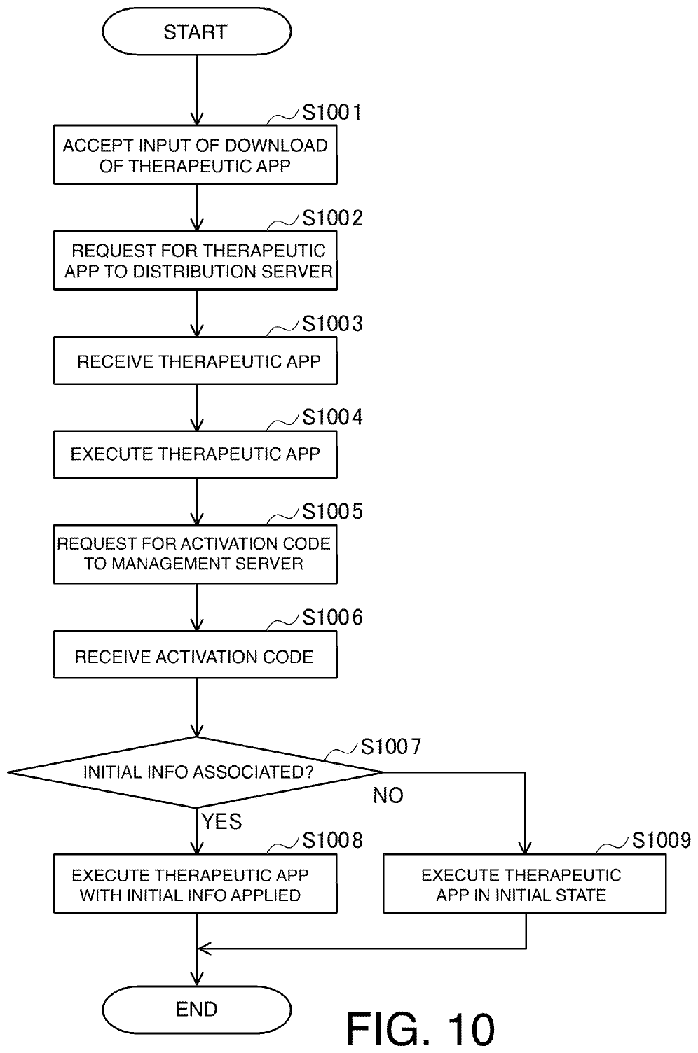

FIG. 10 is a flowchart illustrating an example of the operations of the user terminal 200 to embody the exchanges shown in FIG. 8.

The input unit 220 of the user terminal 200 accepts input from the patient instructing the download of the therapeutic application (step S1001). The input unit 220 communicates the accepted input content to the controller 230. The controller 230 transmits, via the communication unit 210, application request information requesting the specified therapeutic application to the distribution server 300 that stores the therapeutic application in accordance with the communicated input content (step S1002). The application request information includes an application ID for identifying which therapeutic application is being requested and user identification information for identifying the user (patient) requesting the therapeutic application. When the application request information is accepted, an installer (or even an executable program) for the specified therapeutic application is transmitted to the distribution server 300.

The communication unit 210 receives the installer of the therapeutic application transmitted from the distribution server 300 (step S1003). The communication unit 210 communicates the received installer of the therapeutic application to the controller 230. When the installer of the therapeutic application is communicated to the controller 230, the controller 230 executes the installation of the therapeutic application, and the application execution unit 231 executes the therapeutic application (step S1004).

The application execution unit 231 requests an activation code to the management server 100 when it is the first time when the therapeutic application is executed. At this time, the application execution unit 231 also transmits the user identification information to the management server 100. The activation code may be directed to a code that is needed to be entered each time (or periodically) the therapeutic application is executed. The application execution unit 231 transmits code request information requesting an activation code to the management server 100 via the communication unit 210 (step S1005). In response, the management server 100 generates and transmits an activation code for the user terminal 200.

The communication unit 210 receives the activation code for the therapeutic application transmitted from the management server 100 (step S1006). The communication unit 210 communicates the received activation code to the controller 230. The controller 230 then causes the output unit 250 to output the communicated activation code. That is, the activation code is displayed on the monitor of the user terminal 200. The patient then enters the displayed activation code through the input unit 220 to run the therapeutic application. The activation code may be specified to be entered directly into the therapeutic application by the program at the time it is received, rather than by the patient.

If initial information is associated with the activation code (YES in step S1007), the application execution unit 231 executes the therapeutic application with the initial information applied (step S1008). If initial information is not associated with the activation code (NO in step S1007), the application execution unit 231 executes the therapeutic application in the initial state thereof (step S1009).

The operation of the user terminal 200 in which the therapeutic application is obtained and initially started is described above.

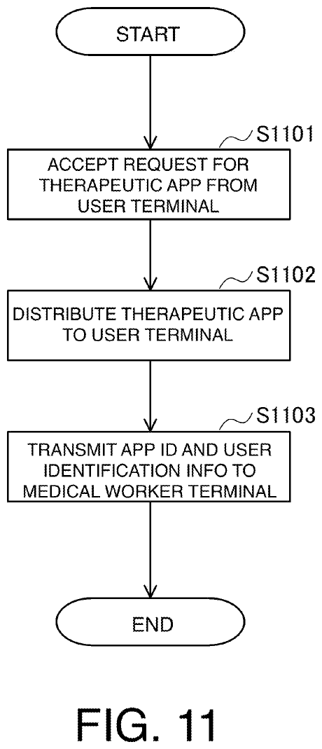

FIG. 11 is a flowchart illustrating an example of the operations of the distribution server 300 to embody the exchanges shown in FIG. 8.

As shown in FIG. 11, the communication unit 310 of the distribution server 300 receives a request signal requesting a therapeutic application from the user terminal 200 (step S1101). The communication unit 310 communicates the received request signal to the controller 330.

When the request signal is communicated, the distribution unit 331 of the controller 330 extracts an application ID that indicates the therapeutic application desired by the patient and included in the communicated request signal. The distribution unit 331 then obtains the installer of the therapeutic application indicating the extracted application ID from the storage 340. The distribution unit 331 distributes the obtained installer of the therapeutic application to the user terminal 200 that has transmitted the request signal via the communication unit 310 (step S1102). This allows the patient to obtain the therapeutic application.

When the request signal is communicated, the controller 330 transmits the application ID of the requested therapeutic application and the user identification information indicating the user (patient) of the user terminal 200 requesting the therapeutic application to the medical worker terminal 500 (step S1103). This can trigger the input of the initial information for the user (patient) by the medical worker at the medical worker terminal 500.

The operation of the distribution server 300 is described above. Regarding the processes in steps S1102 and S1103, the order thereof may be inverted.

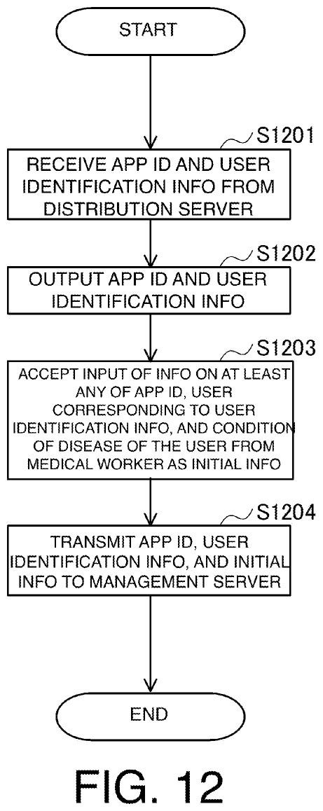

FIG. 12 is a flowchart illustrating an example of the operations of the medical worker terminal 500 to embody the exchanges shown in FIG. 8.

As shown in FIG. 12, the communication unit 510 of the medical worker terminal 500 receives from the distribution server 300 an application ID indicating a therapeutic application and user identification information indicating a user (patient) in need of the therapeutic application (step S1201). The communication unit 510 communicates the received application ID and user identification information to the controller 530.

The controller 530 causes the output unit 550 to output (display) information indicating the received application ID and user identification information (step S1202). This allows a medical worker to recognize which patients desire which therapeutic applications.

The input unit 520 accepts input of initial information regarding at least one of the symptoms that the therapeutic application indicated by the application ID received in step S1201 targets for treatment and the user (patient) indicated by the user identification information (step S1203). The input unit 520 communicates the accepted initial information to the controller 530.

The initial information transmission unit 531 then transmits the initial information entered by the medical worker, the corresponding application ID and user identification information to the management server 100 via the communication unit 510 (step S1204) and terminates the process.

This allows the management server 100 to use the therapeutic application used by each patient with the initial information specified by the medical worker not in the initial state thereof but in a state in which some information has been entered even if the patient has not entered any information. Accordingly, the patient need not go through a complicated input process when using the therapeutic application so that the possibility that the patient will not use the therapeutic application since he/she finds the input process troublesome can be restricted.

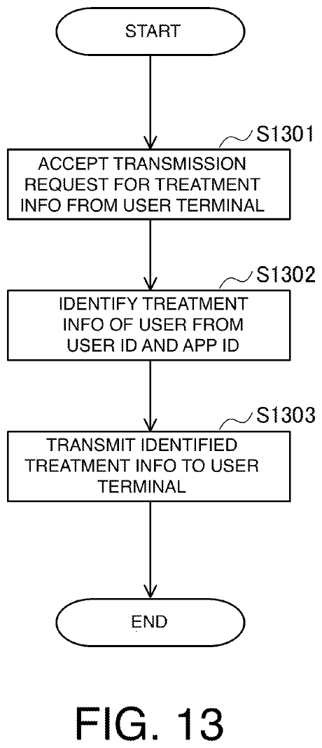

FIG. 13 is a flowchart illustrating an example of the operations of the operation server 600. As shown in FIG. 13, the communication unit 610 of the operation server 600 accepts a request for transmission of treatment information transmitted from the user terminal 200 (step S1301). The treatment information here is directed to information on the treatment presented to the patient by the therapeutic application, and may include, for example, the details of previous treatments performed and their success or failure, the medicines used, information on the behavior of the patient, and various other information. This transmission request may include at least a user ID (patient ID) capable of identifying the user, and may also include a password to view the treatment information. When the communication unit 610 receives a request to transmit the treatment information, it communicates it to the controller 630.

The treatment information provision unit 631 of the controller 630 identifies the patient ID from the communicated transmission request for the treatment information. The treatment information provision unit 631 then identifies the treatment information 641 corresponding to the identified patient ID from the storage 640 (step S1302). The treatment information provision unit 631 then transmits the identified treatment information to the user terminal 200 via the communication unit 610 (step S1303). The treatment information 641 here may be the same as the management information 141 managed by the management server 100.

This allows the operation server 600 to provide the user terminal 200 with information on the treatment of the user. If a password is included in the transmission request for the treatment information from the user terminal 200, the password may be authenticated first in step S1302, and if the password is correct, the treatment information may be identified.

### Display Examples

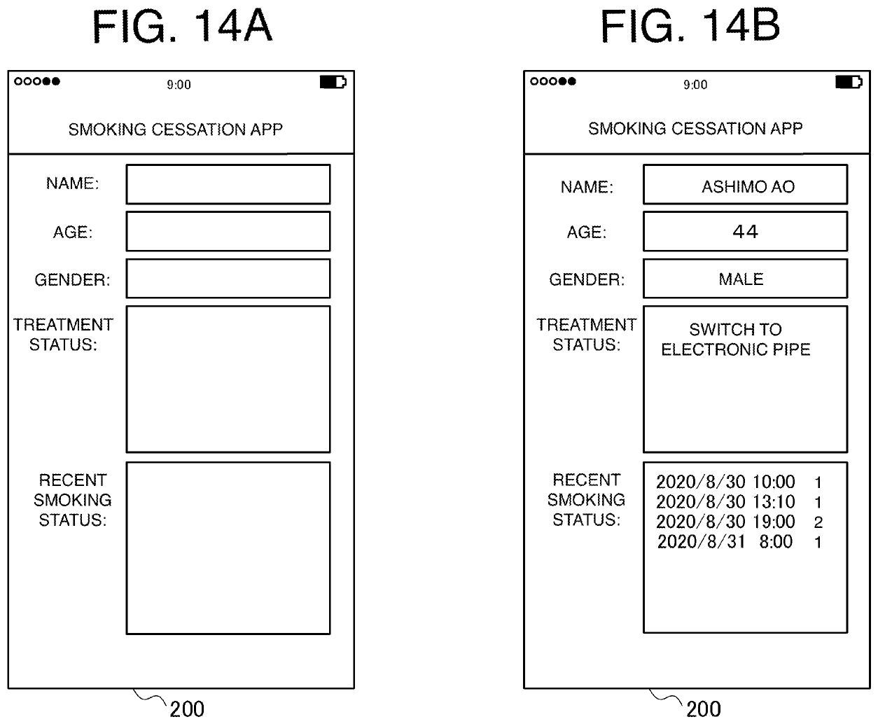

FIGS. 14A and 14B show examples of display of the therapeutic application. FIGS. 14A and 14B are only examples and may be displayed in other forms.

FIG. 14A shows an example of the initial screen of the therapeutic application with no initial information applied, and FIG. 14B shows an example of the initial screen of the therapeutic application with the initial information applied. FIGS. 14A and 14B show an example of displaying a smoking cessation application as a type of therapeutic application, but it is not limited to smoking cessation as the therapeutic application.

As shown in FIG. 14A, without the initial information, i.e., without any information, the patient needs to enter various types of information such as the name and age, which is tiresome. Since the initial input is tiresome, some patients may fail to use the therapeutic application. To reduce such a possibility, in this example, the initial information is applied to start the therapeutic application with various information applied even if the application is initially started as shown in FIG. 14B. Accordingly, the therapeutic application can be easily used since it does not force the patient to go through the tiresomeness for various inputs.

Further, the therapeutic application is directed to an application that provides suggestions to the patient for treatment in accordance with the stage of the treatment. However, without the initial information, an appropriate suggestion may fail to be given to the patient in accordance with the stage of treatment so that a wrong suggestion may be given to the patient from the point of view of the patient. In this example, the patient might then determine the application to be unhelpful and stop using the therapeutic application, however, the application of the initial information allows the suggestion to be appropriate for the patient from the beginning.

### Example of Initial Operation of Therapeutic Application Based on the Presence of Initial Information

An example will be described where the initial information is applied to the therapeutic application so that appropriate suggestions can be given to the patient from the beginning. These operations are described here as the operations of the therapeutic application, but the therapeutic application may also embody the operations described below in collaboration with the management server 100 or the operation server 600.

The therapeutic application may suggest chatbot treatment to the patient. The therapeutic application may also prepare multiple types of chatbots. For example, various types of chatbots may be prepared such as a physician-type chatbot, a nurse-type chatbot, or a chatbot that is set up to suffer from the same disease. Depending on the chatbot, the content of the suggestions and the manner in which suggestions are given to the patient may differ. In the actual world, it is publicly known that the relationship of trust between a patient and a doctor or nurse also involves compatibility, and the same may be applied to the chatbots.

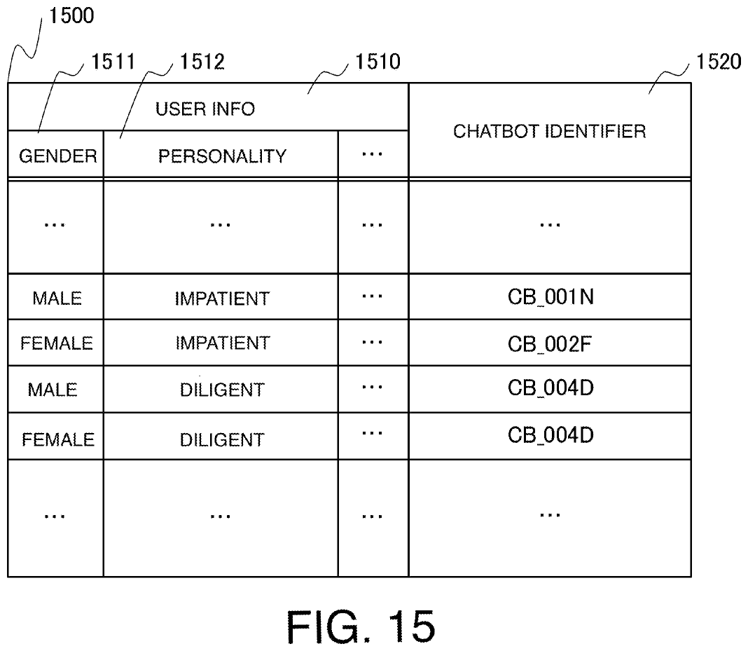

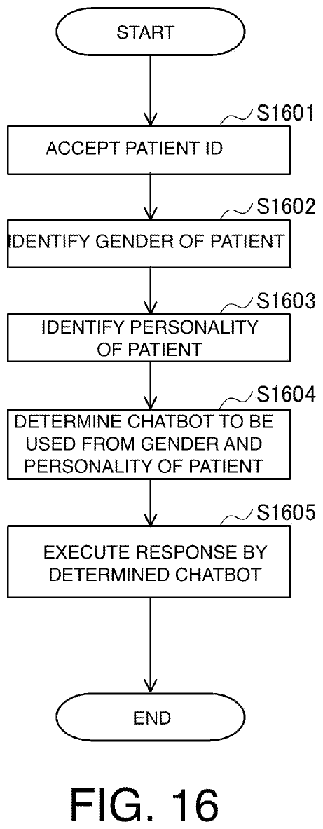

Accordingly, it is desirable to prepare a chatbot that is compatible with the patient from the initial execution to allow the patient to be more positive about the treatment. Accordingly, with reference to FIGS. 15 and 16, a method of selecting a chatbot will be described.

FIG. 15 is a conceptual data diagram showing an example of a chatbot compatibility table to be used when selecting a chatbot.

The chatbot compatibility table 1500 may be stored as a portion of the therapeutic application 241 in the storage 240 of the user terminal 200, or stored in the storage of the management server 100 or the operation server 600.

As shown in FIG. 15, the chatbot compatibility table 1500 is directed to information in which the user information 1510 is associated with the chatbot identifier 1520.

The user information 1510 is directed to information that indicates patient attributes used to determine a compatible (presumed to be compatible) chatbot. The example shown in FIG. 15 shows that the user information 1510 includes, but is not limited to, gender 1511 and personality 1512.

The chatbot identifier 1520 corresponds to information that uniquely identifies each chatbot in the system, and may be a symbol or number assigned by the operator of the management server 100, for example, and may be indicated by a name indicating the characteristics thereof (e.g., strict male physician type).

The chatbot compatibility table 1500 may be a table in which chatbots are determined in accordance with various combinations of patient information. Here, a plurality of different chatbots may be associated with the same combination, and in this case, the chatbot may be randomly determined among the different chatbots.

In the example in FIG. 15, for example, the therapeutic application may use the chatbot indicated by “CB 001N” to give the first suggestion for a patient with the gender of, for example, “male” and the personality of, for example, “short-tempered.” The chatbot for giving the suggestions to the patient may change over time. For example, if the personality of the patient is presumed to have changed, the changed personality may be used to identify the chatbot.

The chatbot compatibility table 1500 may be created by an operator of the management server 100, or it may be created by, for example, the controller 130 of the management server 100, on the basis of the personality of each patient and the aggregate results of aggregating information on chatbots that have been compatible with the personality of the patient (e.g., chatbots that the patient has used for a predetermined period or longer) in the process of various patients using the therapeutic application.

FIG. 16 is a flowchart illustrating an example of operation to determine the chatbot to be used by the therapeutic application (the controller 230 of the user terminal 200) to treat the patient. Although described here as the operation of the user terminal 200, the same process can also be embodied by the management server 100.

As shown in FIG. 16, the controller 230 accepts input of a patient ID (step S1601). This input may be entered from the patient, or information may be accepted as input when the therapeutic application is entered or exited, or when the initial information is applied.

The controller 230 identifies the gender of the patient using the information registered in the therapeutic application (step S1602).

Next, the controller 230 identifies the personality of the patient (step S1603). The controller 230, for example, accesses the management server 100 to obtain information on the personality of the patient that is registered in the management information 141.

The controller 230 determines the chatbot to be used by referring to the chatbot compatibility table 1500 on the basis of the gender and personality of the identified patient (step S1604).

The controller 230 then executes a response (treatment suggestion) by the determined chatbot as a part of the treatment by the therapeutic application (step S1605).

As described above, the therapeutic application is capable of determining which chatbot to use first and using it for treatment suggestions. This chatbot is recognized as compatible with the patient, allowing the patient to feel comfortable using the therapeutic application from the beginning.

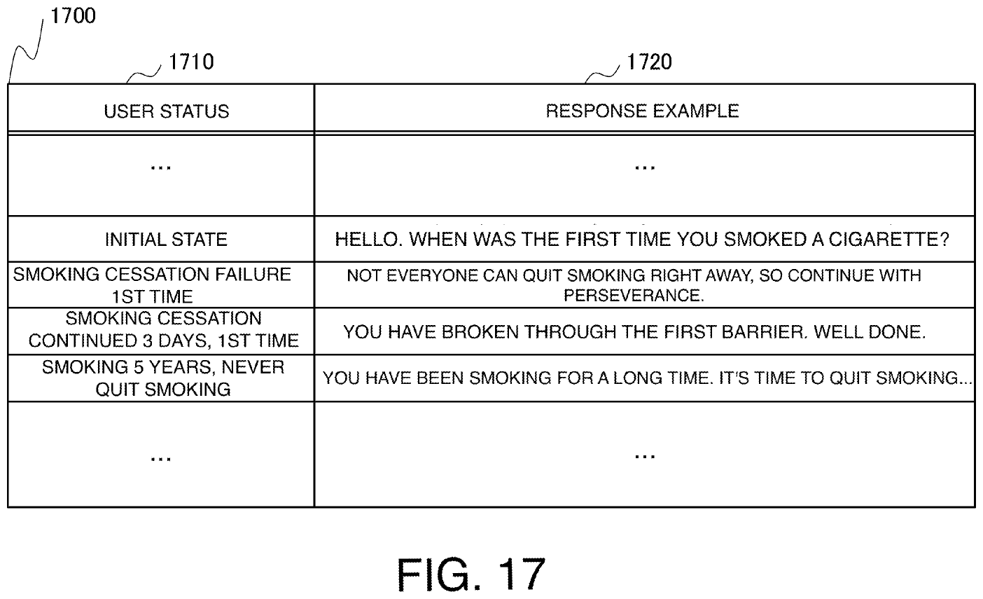

Next, with reference to FIGS. 17 through 19, an example where a chatbot can respond appropriately in accordance with the stage of treatment will be described.

The therapeutic application may store a response table 1700 shown in FIG. 17. The response table 1700 may be stored in the management server 100 or the operation server 600, or it may be referred to by accessing the server from the therapeutic application of the user terminal 200.

The response table 1700 corresponds to a table in which user status 1710 and response example 1720 are associated with each other.

The user status 1710 corresponds to information indicating the status of the patient. In FIG. 17, as an example, information indicating the stage of the treatment is shown as the user status 1710.

The response example 1720 corresponds to information that shows an example of a response to be performed by the chatbot when the user is in the corresponding user status. Although FIG. 17 shows an example of one response associated with user status 1710, multiple response examples may be associated, and, in this example, one of them may be selected to be the response. The response shown in the response example 1720 may change tone depending on the responding chatbot. For example, the chatbot may respond in a stern tone if it corresponds to a physician, or in a more familiar tone if it corresponds to a fellow chatbot suffering from the same disease.

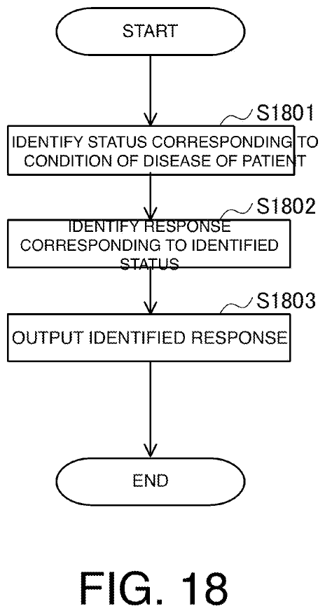

FIG. 18 is a flowchart illustrating an example of operation in which the controller 230 of the user terminal 200 determines the response of the chatbot as a part of execution by the therapeutic application.

As shown in FIG. 18, the controller 230 identifies the status corresponding to the condition of the disease of the patient by referring to the information stored in the therapeutic application or the treatment information registered in the operation server 600 (step S1801).

Next, the controller 230 identifies the corresponding response by referring to the identified status of the patient and the response table 1700 (step S1802).

The controller 230 then causes the chatbot in use to output the identified response (step S1803).

As described above, the therapeutic application allows the chatbot, which is compatible with the user, to give a response and suggestion appropriate to the condition of the patient.

Although an example of response table 1700 is shown here for clarity, the response by the chatbot may be based on the conditions of the patient (age, gender, personality, mental state, and treatment state, for example) and a learning model that has learned suggestions and responses in accordance with the conditions to determine the response content.

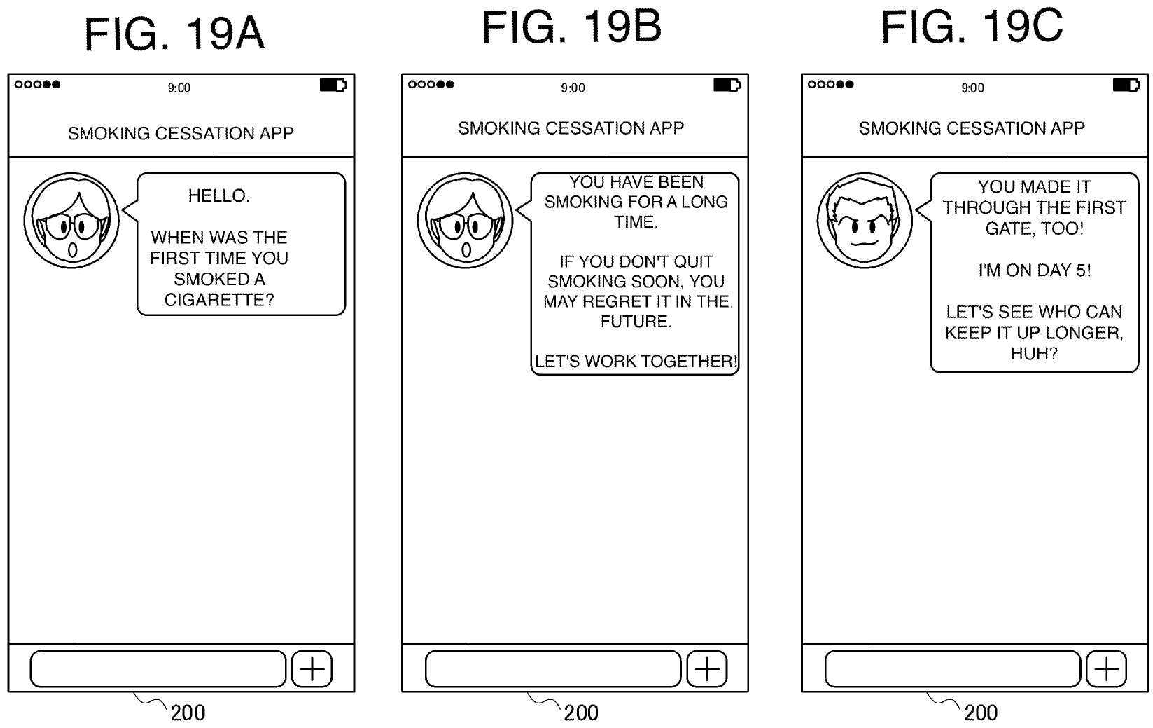

FIGS. 19A to 19C show examples of displays of responses by the chatbots, and the screenshots showing examples of responses for different patient conditions and chatbots.

FIG. 19A is an example display showing an example response with no initial information applied. Since the chatbot makes a response without information, it also needs to learn various basic information. In contrast, in FIG. 19B, the initial information has been entered, and thus the chatbot makes a statement such that it understands that the patient has been smoking for a long time. FIGS. 19A and 19B show examples of responses by the chatbots of physicians. In contrast, some patients can do their best if they have a fellow with a similar condition fighting against a similar disease, and FIG. 19C shows an example of a response by a chatbot for such a patient. As described above, a variety of different types of chatbots are prepared so that the therapeutic applications capable of accommodating different types of patients can be provided. In addition, among those chatbots, the chatbot that is estimated to be the most suitable for the patient can be used to increase the rate of continued use of the therapeutic application by the patient.

The response of the chatbot may be determined by the management server 100 or the operation server 600, and the management server 100 or the operation server 600 may be configured to communicate the determined response to the user terminal 200 so that the chatbot responds as specified on the user terminal 200.

### Variation

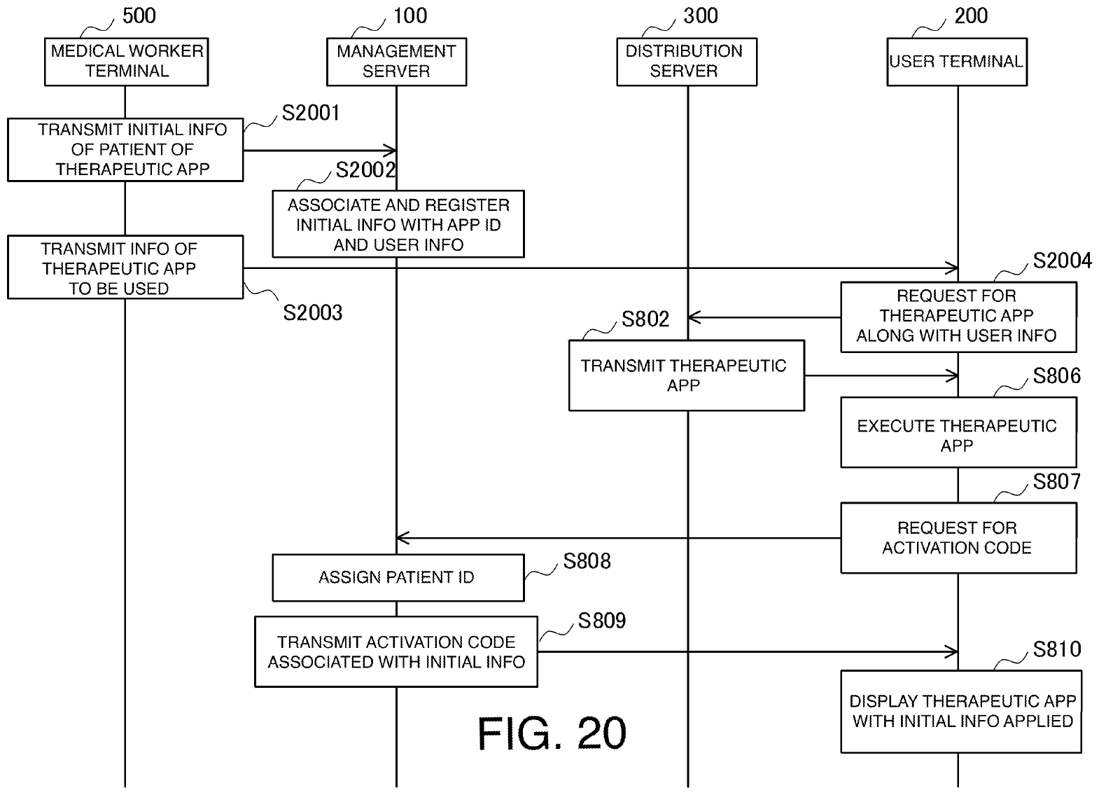

In the example above, as shown in the sequence diagram in FIG. 8, an example of the use of the therapeutic application with which the initial information from the user (patient) side approach is associated is described. However, examples of the operation of therapeutic applications by the therapeutic application distribution system are not limited to this. As shown in FIG. 20, the patient may be allowed to use the therapeutic application in a manner that approaches the user (patient) from the medical worker.

FIG. 20 is a sequence diagram illustrating an example of the exchanges of the devices associated with the therapeutic application distribution system in this variation. The medical worker using the medical worker terminal 500 enters the initial information on the user (patient) to the medical worker terminal 500 when a user (patient) where the medical worker wishes to allow him/her to use the therapeutic application is present. The medical worker terminal 500 then transmits the entered initial information, user identification information indicating the user (patient) that is to use the initial information, and an application ID indicating the therapeutic application to be used to the management server 100 (step S2001).

Upon receiving the application ID, user identification information, and initial information, the management server 100 associates and stores them with each other in the storage 140 (step S2002). This process is the same as the process of step S805 in FIG. 8 and step S902 in FIG. 9.

The medical worker terminal 500 then transmits information on the therapeutic application to be used to the user terminal 200 (step S2003).

Although it is assumed here that the notification is transmitted from the medical worker terminal 500 to the user terminal 200, this may be communicated directly and verbally from the medical worker at the medical worker terminal 500 to the user (patient) at the user terminal 200.

The processes after step S802 are the same as those described using FIG. 8, and thus the description thereof is omitted. Regarding the processes of steps S2001 and S2002 and the process of step S2003, the order thereof may be optionally changed.

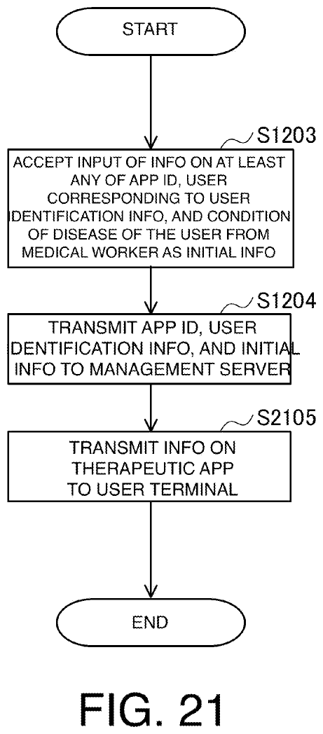

FIG. 21 is a flowchart illustrating an example of the operations of the medical worker terminal 500 to embody the exchanges shown in FIG. 20. As can be understood by comparing FIG. 21 with FIG. 12, in this variation, steps S1201 and S1202 in FIG. 12 are not performed. As shown in step S2105, the controller 530 of the medical worker terminal 500 then transmits information (application ID) on the therapeutic application to the user terminal 200 (step S2105). This information on the therapeutic application may include information on the URL to download the therapeutic application.

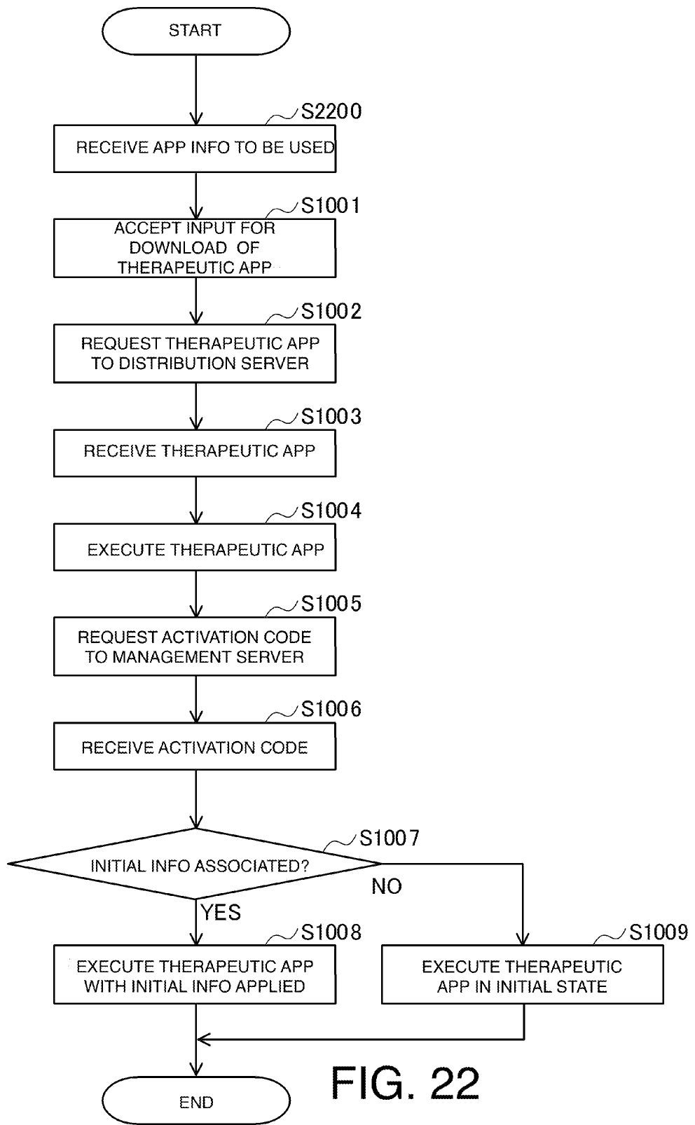

FIG. 22 is a flowchart illustrating an example of the operations of the user terminal 200 to embody the exchanges shown in FIG. 20. As can be understood by comparing FIG. 22 with FIG. 10, in this variation, the user terminal 200 receives information on the therapeutic application from the medical worker terminal 500 (step S2200) before executing the processes in step S701 and subsequent steps. This allows the user of the user terminal 200 to recognize the existence of the therapeutic application and download the therapeutic application. Steps S701 and thereafter are the same as those in FIG. 7, and thus the description thereof is omitted.

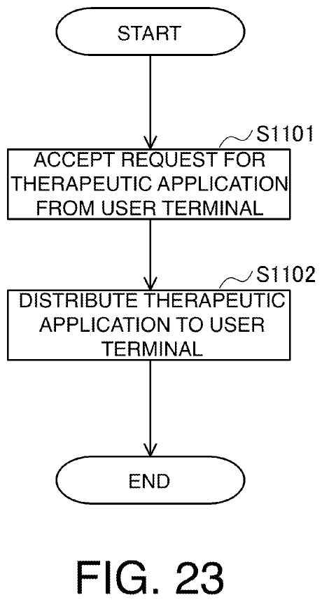

FIG. 23 is an example of the processing of the distribution server 300 according to a variation. As can be understood by comparing FIG. 23 with FIG. 11, this variation differs in that the process of step S1103 is not performed, and the other processes are common.

As described above, the therapeutic application distribution system may approach the patient from the medical worker's side to allow the patient to use the therapeutic application.

### Supplement

In the example above, as a method of executing the therapeutic application by the user terminal 200, the processors of the devices including the user terminal 200 execute a generation program, for example, to generate the therapeutic application and initial information, and the therapeutic application is started to be used with the initial information applied to it, but this may be by a logic circuit (hardware) or dedicated circuit formed in an integrated circuit (integrated circuit (IC) chip and large scale integration (LSI), for example) in the device. The functional units may be embodied by one or more integrated circuits, and the functions of a plurality of functional units shown in the embodiment above may be by a single integrated circuit. The LSI is sometimes referred to as a VLSI, Super LSI, and Ultra LSI, for example, depending on the different degree of integration.

The above-described control program may be recorded on a recording medium that is readable by the processor, and the recording medium may be a “non-temporary tangible medium,” such as a tape, disk, card, semiconductor memory, and programmable logic circuit. The above-described management program may be supplied to the above processor via any transmission medium capable of transmitting the management program (such as a communication network or broadcast wave). That is, the system may be configured to download and execute the management program from the network using, for example, a smart phone or other information processing device. The present disclosure may also be embodied in the form of a data signal embedded in a carrier wave, where the above-described management program is embodied by electronic transmission.

The above program may be implemented using, for example, a scripting language such as ActionScript or JavaScript (registered trademark), an object-oriented programming language such as Objective-C, Java (registered trademark), C++, Python, or R, but it is not restricted to them.

### INCORPORATION BY REFERENCE

This application is a continuation application of International Application No. PCT/JP2021/034152, filed on Sep. 16, 2021, which claims priority of Japanese Patent Application No. 2020-179114, filed on Oct. 26, 2020, the contents of which are hereby incorporated by reference in their entirety.

What is claimed is:

1. A therapeutic application distribution system comprising: a server including:
a first reception unit that receives, from an information processing terminal of a patient to be treated by a therapeutic application, user identification information identifying the patient, an application ID identifying the therapeutic application, and request information requesting an activation code to execute the therapeutic application;
an acceptance unit that accepts input of initial information on the user identification information and a disease to be treated by the therapeutic application from a medical worker, and associates and stores the user identification information with the initial information in a storage;
a generator that generates an activation code for executing the therapeutic application corresponding to the user identification information; and
a first transmission unit that transmits the activation code generated by the generator to the information processing terminal of the patient; and
an information processing terminal including:
an application acquisition unit that acquires a therapeutic application in an initial state;
a second transmission unit that transmits to the server the user identification information, the application ID identifying the therapeutic application acquired by the application acquisition unit, and the request information requesting the activation code to execute the therapeutic application;
a second reception unit that receives the activation code from the server;
an execution unit that executes the therapeutic application; and
an output unit that outputs information based on the therapeutic application, wherein
the execution unit executes the therapeutic application with the initial information applied based on input of the activation code at an initial execution of the therapeutic application.

2. The therapeutic application distribution system according to claim 1, wherein
the initial information includes an age of the patient,
the therapeutic application makes a suggestion for treatment of the disease, and
the output unit outputs a suggestion based on the age of the patient as information based on the therapeutic application.

3. The therapeutic application distribution system according to claim 1, wherein
the initial information includes gender of the patient,
the therapeutic application outputs a suggestion for treatment of the disease via one of a plurality of characters, and
the output unit outputs the suggestion as information based on the therapeutic application by a character suitable for the gender of the patient among the characters.

4. The therapeutic application distribution system according to claim 1, wherein
the initial information includes historical information on treatment for the disease of the patient, and
the output unit outputs information indicating a history of the treatment of the patient based on the history information as information based on the therapeutic application.

5. The therapeutic application distribution system according to claim 4, wherein
the output unit outputs, as information based on the therapeutic application, a suggestion for treatment in accordance with a progress of the disease based on the historical information of the patient.

6. The therapeutic application distribution system according to claim 4, wherein the historical information is an electronic medical record of the patient.

7. The therapeutic application distribution system according to claim 1, wherein
the initial information is information on personality of the patient, and
the output unit outputs a suggestion based on the personality of the patient as information based on the therapeutic application.

8. The therapeutic application distribution system according to claim 7, wherein
the server includes a provision unit that provides an input form for requesting the activation code to the information processing terminal,
the input form includes a questionnaire for the patient,
the second transmission unit further transmits a response of the patient to the questionnaire,
the first reception unit further receives the response of the patient, and
the initial information includes information based on the response of the patient to the questionnaire as information on the personality of the patient.

9. The therapeutic application distribution system according to claim 1, wherein
the initial information includes information on a suggestion for treatment performed on the patient by the medical worker, and
the output unit outputs a suggestion the same as the suggestion as information based on the therapeutic application.

10. A therapeutic application distribution method comprising:
an application acquisition step of acquiring a therapeutic application in an initial state by an information processing terminal;
a first transmission step of transmitting, by the information processing terminal, to a server user identification information, an application ID identifying a therapeutic application acquired in the application acquisition step, and request information requesting an activation code to execute the therapeutic application;
a first reception step of receiving, by the server from the information processing terminal of a patient to be treated by the therapeutic application, user identification information identifying the patient, an application ID identifying the therapeutic application, and request information requesting an activation code to execute the therapeutic application;
an acceptance step of accepting, by the server, input of initial information on the user identification information and a disease to be treated by the therapeutic application from a medical worker, and associating and storing the user identification information with the initial information in a storage;
a generation step of executing, by the server, the therapeutic application corresponding to the user identification information;
a second transmission step of transmitting, by the server, the activation code generated in the generation step to the information processing terminal of the patient;
a second reception step of receiving, by the information processing terminal, the activation code from the server;
an execution step of executing, by the information processing terminal, the therapeutic application; and
an output step of outputting, by the information processing terminal, information based on the therapeutic application, wherein
in the execution step, the therapeutic application is executed with the initial information applied based on the input of the activation code at an initial execution of the therapeutic application.

11. A non-transitory computer readable medium storing therein a therapeutic application distribution program causing a computer to embody:
a reception function that receives, from a terminal of a patient to be treated by a therapeutic application, user identification information identifying the patient, an application ID identifying the therapeutic application, and request information requesting an activation code to execute the therapeutic application;
an acceptance function that accepts input of initial information on the user identification information and a disease to be treated by the therapeutic application from a medical worker, and associates and stores the user identification information with the initial information in a storage;
a generation function that executes the therapeutic application corresponding to the user identification information; and
a transmission function that transmits the activation code generated by the generation function to the terminal of the patient.
